(19) United States
(12) Patent Application Publication
(10) Pub. No.: US 2010/0137575 A1
(43) Pub. Date: Jun. 3, 2010

(54) UNIVERSAL BIOLOGICAL SAMPLE PROCESSING

(76) Inventors: D. Michael Connolly, Rochester, NY (US); Vera Tannous, Penfield, NY (US); Richard S. Murante, Rochester, NY (US); Charles DeBoer, Ithaca, NY (US)

Correspondence Address: HISCOCK & BARCLAY, LLP, 2000 HSBC PLAZA, 100 Chestnut Street, ROCHESTER, NY, 14604-2404, US

(21) Appl. No.: 12/626,294

(22) Filed: Nov. 25, 2009

Related U.S. Application Data
(60) Provisional application No. 61/119,597, filed on Dec. 3, 2008.

(51) Int. Cl. C07H 1/06

(52) U.S. Cl. 536/25.41

(57) ABSTRACT

A method for preparing a sample by utilizing a mechanical force in the presence of a size stabilizer to break apart the sample to obtain nucleic acid molecules in a usable size range.

### CROSS-REFERENCE TO RELATED APPLICATIONS

This application claims priority from U.S. Provisional Patent Application Ser. No. 61/119,597, filed Dec. 3, 2008, the contents of which are hereby incorporated by reference.

The U.S. Government has a paid-up license in this invention and the right in limited circumstances to require the patent owner to license others on reasonable terms as provided for by the terms of one or more of the following Grant Award Nos. DMI-0450472 and IIP-0450472 awarded by National Science Foundation, Contract No. W81XWH-07-2-0109 awarded by US Army Medical Research and Material Command, Contract Nos. W911NF-06-1-0238 and W911NF-09-C-0001 awarded by US Army RDECOM ACQ CTR.

### FIELD OF THE INVENTION

This invention relates to a process for preparing nucleic acid molecules from biological samples. More particularly, this invention relates to a method for preparing samples by breaking down a biological sample in the presence of a size stabilizer to obtain nucleic acid molecules within a usable base pair range.

### BACKGROUND OF THE INVENTION

Nucleic acid based identification of biological material first requires isolation of the nucleic acid molecules (NAMs) from the sample. In order for a system to effectively and efficiently meet the users needs, a universal sample preparation process is required. Current sample preparation processes are laborious, time consuming and require laboratory capability. To remain universal, the process must be able to handle a wide variety of input materials. This includes, but is not limited to, viruses, spores, organisms, bacteria and medical diagnostic materials, such as blood, tissue, saliva, urine and feces.

There is continuing interest to improve testing methodologies and decrease time demands on clinical laboratories. Particular testing requires that a sample be broken down to extract nucleic acid molecules such as DNA or RNA.

It is estimated that about 30 million molecular diagnostic tests took place in US medical facilities in 2007. This figure is expected to increase to 67 million in 2009. Many, if not all of these assays, could benefit from a rapid sample preparation process that is easy to use, requires no operator intervention, is cost effective and is sensitive to small size samples.

The use of molecular diagnostics and gene sequencing in research and medical diagnostics are rapidly growing. Molecular techniques provide higher levels of specificity and sensitivity than antibody methods, Genetic sequencing allows for the collection of large amounts of information not previously available. However, sample preparation is a major cost component of running PCR, real-time PCR, gene sequencing analysis and hybridization testing. In addition, it delays test results and limits the ability to run these assays to laboratories with well trained personnel.

Bead beating has been used for years to isolate nucleic acid molecules from samples. Bead beating is the agitation, usually by ultrasound, of micron size glass beads added to the sample. It is a robust approach which is well suited for use with solids like spores or tissue.

Bead beating has several drawbacks. On one hand, if the sample is treated too long, or at too high a power level, only short fragments less than 100 bases long are produced. On the other hand, if the sample is treated to brief, low power agitation, a low yield of nucleic acid is produced, along with a wide range of fragment sizes. When particular size ranges of nucleic acids are needed, gel electrophoresis of the sample is sometimes employed, cutting the gel sections with the correct size ranges out of the finished gel and extracting the nucleic acid fragments from the gel. This process is both slow and tedious.

In running biological and chemical tests it is often desired to obtain a usable size range of nucleic acid molecules and to concentrate and retain the desired analyte. Concentrating the sample can be a difficult process. Traditional methods for concentrating a biological sample include filtering, rinsing, centrifuging and/or reaction chemistry. Often these steps cannot be preformed in a single processing chamber and require the sample to be transferred to other devices or chambers.

Magnetic nanoparticles are particles which are attracted to a magnetic field. By attaching a magnetic nanoparticle to nucleic acid polymers and applying a magnetic field to a sample, the nucleic acid polymers can be moved to a desired location, thereby concentrating a portion of the sample with the nucleic acid polymers. The sample can then be drawn from the concentrated portion yielding a high amount of nucleic acid polymers.

Applying a magnetic field further allows for manipulating the nucleic acid polymer. For example, by holding a nucleic acid polymer steady a rinse can be applied without washing away the nucleic acid polymer.

Therefore, there is a need for a method to prepare nucleic acid samples from any source in a desired size range, rapidly and economically.

### SUMMARY OF THE INVENTION

The present invention describes a novel sample preparation approach which is universal for numerous biological sample types. The process breaks down cells, tissue or other materials to release nucleic acid molecules. During this process the nucleic acid molecules are also broken down to manageable sizes. In one embodiment the nucleic acid molecules are concentrated and cleaned. Particles can be held in a flow to be washed. The nucleic acid molecules are then eluted from particles using a buffer or heat.

Providing an universal sample preparation process can greatly reduce the costs and increase the reproducibility. For example, automated gene sequencing systems require extensive processing of samples to prepare DNA for analysis. Most DNA sequencing approaches use an in vitro cloning step to amplify individual DNA molecules. Emulsion PCR isolates individual DNA molecules along with primer-coated beads in aqueous droplets within an oil phase. PCR then coats each bead with clonal copies of the DNA molecule followed by immobilization for later sequencing. Emulsion PCR is used in the methods by Marguilis et al. (commercialized by 454 Life Sciences), Shendure and Porreca et al. (also known as “polony sequencing”) and SOLiD sequencing, (developed by Agencourt, now Applied Biosystems). Another method for in vitro clonal amplification is bridge PCR, where fragments are amplified upon primers attached to a solid surface. The single-molecule method developed by Stephen Quake's laboratory (later commercialized by Helicos) skips this amplification step, directly fixing DNA molecules to a surface.

Since both sonicated DNA fragments can contain single-stranded ends, most procedures include a step to end-repair the DNA prior to ligation into blunt-ended vectors (10,11). A combination of T4 DNA polymerase and Klenow DNA polymerase are used to “fill-in” the DNA fragments by catalyzing the incorporation of complementary nucleotides into resultant double-stranded fragments with a 5′ overhang. Additionally, the single-stranded 3′-5′ exonuclease activity of T4 DNA polymerase is used to degrade 3′ overhangs. The reactions included the two enzymes, buffer, and deoxynucleotides and are incubated at about 37° C. The fragments are concentrated by ethanol precipitation followed by resuspension in kinase buffer, and phosphorylation using T4 polynucleotide kinase and rATP. The polynucleotide kinase is removed by phenol extraction and the DNA fragments are concentrated by ethanol precipitation, dried, resuspended in buffer, and ligated into blunt-ended cloning vectors. Since, a significant portion of sonicated DNA fragments are easily cloned without end-repair or kinase treatment, these two steps can be combined without significantly affecting the overall number of resulting transformed clones.

Currently, following fragment end-repair, the DNA samples are electrophoresed on a preparative low-melting temperature agarose gel versus a size marker, and after appropriate separation, the fragments in the size range from 1-2 Kbp and 2-4 Kbp are excised and eluted separately from the gel. Alternatively, the fragments can be purified by fractionation on a spin column such as a Sephacryl S-500.

The sample preparation process of the instant invention can prepare fragments of DNA and RNA in a size range of between 100 and 10,000 base pairs. The exact distribution of sizes can be varied by changing concentrations of surfactants, the surfactants used or the frequency of sonication. The ability to produce fragments in the desired size range obviates the need for electrophoresis or column isolation. This also increases the overall yield of useful fragments by eliminating the need for addition purification steps.

In one form, the invention comprises a sample preparation chamber for breaking apart a sample to obtain nucleic acid molecules. A mechanical force is applied in the presence of a size stabilizer to both break apart the sample and obtain nucleic acid fragments in the desired size range.

The invention comprises, in one form thereof, a method for utilizing magnetic nanoparticle containing a target analyte binding element to bind the magnetic nanoparticle to a target analyte. The magnetic nanoparticle is capable of being manipulated within a magnetic field. As the magnetic nanoparticle is bound to the target analyte the target analyte is indirectly manipulated by the application of a magnetic field.

In one embodiment, the magnetic nanoparticles are released from the nucleic acid molecule via the application of heat. Temperatures around 95° C. have been shown to effectively release the magnetic nanoparticles. In another embodiment the magnetic nanoparticles are released from the nucleic acid molecule via an elution solution. The elution solution may be a detergent or salt. In a preferred embodiment, the elution solution contains phosphates or citrates. In one embodiment the elution solution is a potassium or sodium phosphate or citrate.

It is an object of the invention to prepare nucleic acid samples within a desired size range.

One advantage of the invention, is a high yield of nucleic acid from the sample preparation.

Another advantage of the invention, is that it can be used with any nucleic acid sample source, including animal tissue, bacterial cells, spores, insects, plants, and viral cells.

Yet another advantage of the invention, is that the nucleic acid produced is pure and clean, without contamination by other biological materials such as proteins, lipids, and cellular debris.

An even further advantage of the invention, is that the sample preparation process generates a high overall yield because most of the fragments are in a usable size range.

Another advantage of the present invention is that in one embodiment the utilization of magnetic nanoparticles allows for sample concentration by applying a magnetic field without additional processing steps.

### BRIEF DESCRIPTION OF THE DRAWINGS

The present invention is disclosed with reference to the accompanying drawings, wherein:

FIG. 1 demonstrates the effective release of nucleic acid molecules from the lysis of spores using ultrasonic bead beating with size stabilizer;

FIG. 2 demonstrates nucleic acid molecules isolated from fruit flies and that the addition of a size stabilizer in lanes 2 and 3 protect the nucleic acid molecules from over shearing, whereas the samples without the denaturants were sheared to a level well below 100 base;

FIG. 3 shows that using this process the nucleic acid molecules from a wide variety of different samples can be treated with the same power levels and time of sonication to give the same size distribution of fragments;

FIG. 4 demonstrates the nucleic acid molecule isolation obtained from using tissue from the ear of a cow;

FIG. 5 demonstrates the nucleic acid molecule isolation obtained from using fruit flies contaminated with soil;

FIG. 6 is a graphical representation showing the release of the nucleic acid molecules from the magnetic particles;

FIG. 7 demonstrates purified DNA recovered from fruit flies;

FIG. 8 demonstrates DNA recovered from fruit flies using various buffers;

FIG. 9 demonstrates the recovery of nucleic acid molecules from yeast, grass and blueberries.

FIG. 10 demonstrates the recovery of nucleic acid molecules from e-coli and that longer sonication times do not change the size distribution; and

FIG. 11 is a graphical representation of DNA recovery from increasing volumes of a bacterial cell culture using the instant invention, the commercial Qiagen kit for DNA recovery and the textbook Phenol/Chloroform method.

Corresponding reference characters indicate corresponding parts throughout the several views. The examples set out herein illustrate several embodiments of the invention but should not be construed as limiting the scope of the invention in any manner.

### DETAILED DESCRIPTION

A mechanical force is applied to a biological sample to break down the sample to release nucleic acid molecules. A size stabilizer is present to obtain nucleic acid molecules within a usable size range. In one embodiment, the sample material is shredded with high speed nano-particles utilizing sonication. This process breaks down cells, tissue or other materials to release nucleic acid molecules. It is understood that the mechanical force can be any force suitable for tearing apart the sample to release the nucleic acid molecules. Suitable mechanical forces include, but are not limited to sonication, nebulization or homogenization. In one embodiment, the nucleic acid molecules are reduced to sizes between 200 and 10,000 base pairs in length. In another embodiment the nucleic acid molecules are reduced to sizes between 300 and 3,000 base pair in length. In another embodiment the nucleic acid molecules are reduced to sizes between 400 and 2,000 base pair in length. In another embodiment the nucleic acid molecules are reduced to sizes between 200 and 500 base pair in length. It is understood that the desired base pair length will vary depending on the downstream sample processing technique. Sample processing techniques include, but are not limited to hybridization, PCR, real-time PCR, reverse transcription-PCR, “lab-on-a-chip” platforms and DNA sequencing.

Biological samples include all biological organisms which contain nucleic acids. Including but not limited to bacteria, spores, blood, tissues, fungi, plants and insects.

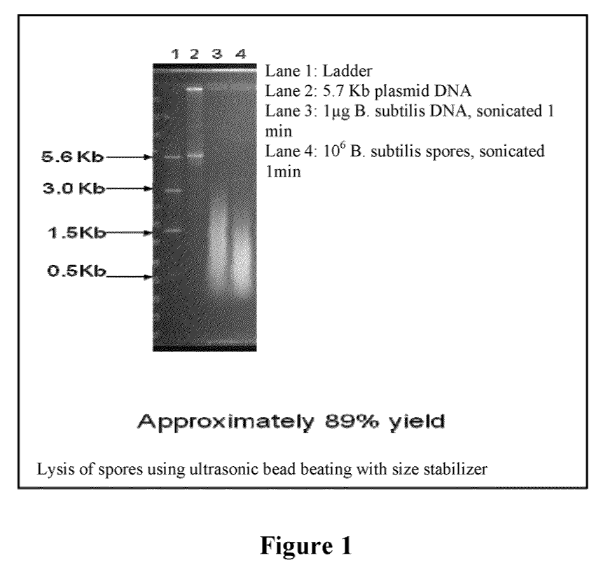

Bead beating is a process to isolate nucleic acid molecules from samples. It is a robust approach which is well suited for use with spores or tissue samples. In bead beating, glass beads of about 100 microns in diameter are used to crush the sample to release the nucleic acid molecules. The particles are moved using an ultrasonic source. FIG. 1 demonstrates the effective release of nucleic acid molecules from spore samples.

To determine efficiency of spore lysis, the maximum amount of nucleic acid output expected from the spores was estimated and compared to the amount measured on the gel in FIG. 1. Utilizing this technique, the method provided an estimate of 85-90% efficiency. Alternatively, spore lysis efficiency can be measured by determining spore survival after sonication. As shown in Table 1, based upon survival assays, the efficiency after two minutes of sonication during experiments was 86% of spores were opened.

TABLE 1
Efficiency of spore lysis as determined
by spore survival (Spore Basis)

| Sonication time | | # spores survived | % efficiency |
|---|---|---|---|
| No sonication | | 235 | |
| 30 | sec. | 105 | 55% |
| 1 | min. | 61 | 74% |
| 2 | min. | 32 | 86% |

Bead beating with sonication however, has had a drawback in that the nucleic acid molecules are degraded during the lysis step. The ultrasonic bead beating shears the nucleic acid molecules to short fragments that are no longer usable. For most uses, fragments need to be larger than 100 bases long. Bead beating often results in fragments much less than 100 bases long.

By utilizing a size stabilizer in solution with the sample the nucleic acid molecules can be protected to limit the minimum size achievable to more desirable base pair length. The addition of size stabilizers in the sample preparation results in a high yield of nucleic acids of limited size range. The size stabilizers include detergents, surfactants, polymers, salts and soaps.

Other size stabilizers of this invention include chaotropic salts such as guanadium thiocyanate. Such salts are known to disrupt the normal folding of proteins associated with nucleic acids, thereby releasing the nucleic acids in free form.

Suspension of the biological sample is done by mixing with a buffer. To retain the desired sample size the buffer serves as a size stabilizer. The size stabilizer is a water solution which may contain salts, detergents, co-solvents or polymers. The size stabilizer prevents the subsequent shearing step from producing fragments of nucleic acid molecules that are too small to be useful in operations such as hybridization, sequencing and polymerase chain reaction (PCR) amplification. For hybridization, fragments of nucleic acid molecules that are smaller than about 18 base pairs lose specificity and are unstable at ambient temperatures. For genetic sequencing and PCR applications, nucleic acid molecule fragments from about 200 to about 500 base pairs are desirable. Use of a pure water buffer gives nucleic acid molecule fragments less than about 100 base pairs, which are too small for many applications.

Use of the size stabilizer allows the gathering of nucleic acid molecule fragments in a desired base pair range. In traditional bead beating processes the mechanical shearing force is turned off after a particular time to maximize the amount of nucleic acid molecule fragments in the desired base pair range. However, because the process is time sensitive a large range of base pair lengths remain present in the sample. By utilizing a size stabilizer the base pair length of most of the sample can be fragmented to the desired base pair range. In one embodiment, at least 60% of the nucleic acid molecule fragments are within 50% of the length of the median nucleic acid molecule fragment base pair length in the sample. Said another way, if the median nucleic acid molecule fragment has 400 base pairs, 60% of the sample would have between 200 and 600 base pairs. In another embodiment, at least 75% of the nucleic acid molecule fragments are within 50% of the length of the median nucleic acid molecule fragment base pair length in the sample. In yet another embodiment, at least 75% of the nucleic acid molecule fragments are within 30% of the length of the median nucleic acid molecule fragment base pair length in the sample.

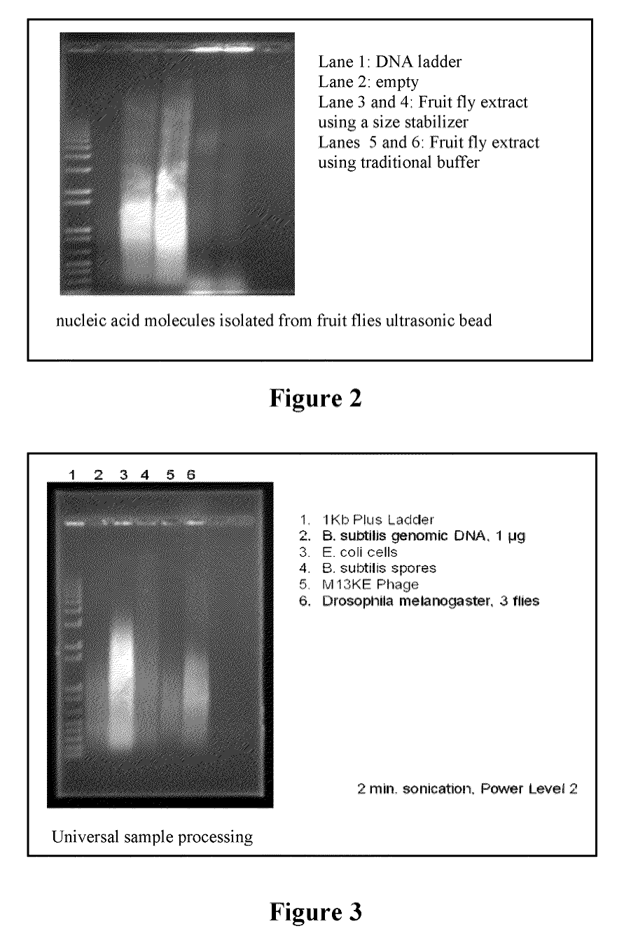

Without a size stabilizer present, the nucleic acid molecules tend to degrade when applying a mechanical force such as sonication. The ultrasonic bead beating with a size stabilizer present shears the nucleic acid molecules into short fragments that are less than 100 bases long (See FIG. 2, lanes 5 and 6). For most applications, fragments need to be larger than 100 bases. As shown in FIG. 2, a series of tests were performed to sonicate purified DNA and RNA sheared polymers to no smaller than 400 bases, even under lengthy sonication times. In complex samples, nucleic acid molecules stick to membranes and proteins while continuing to break down to smaller fragments. To overcome this problem, the lysis buffer is modified to contain a size stabilizer such as a detergent like sodium dodecyl sulfate (SDS). As shown in FIG. 2, the addition of the size stabilizer shown in lanes 3 and 4 protects the nucleic acid molecules from over shearing. The samples without the size stabilizer were sheared to well below 100 bases, as shown in lanes 5 and 6.

The size stabilizer is contained in a protective buffer solution. It is understood that the protective buffer may contain numerous size stabilizers to achieve the desired base pair range. Salts which may be used in the protective buffer include, sodium phosphate, guanidinium hydrochloride and dextran sulfate. The protective buffer may further contain detergents such as sodium dodecyl sulfate, sodium dodceyl benzene sulfate, and polyethyleneglycol. Many commercial anionic surfactants such as Alkanol XC may also be used. In another embodiment the protective buffer includes co-solvents. Co-solvents include dipole aprotic solvents such as dimethylsulfoxide, dimethyl formamide, dimethylacetamide, hexamethyl phosphoramide and tetramethylurea. In another embodiment the protective solution contains polymers such as poly vinyl alcohol, polyethylenimine, poly acrylic acid and other polymeric acids. The concentration of the salts, detergents, co-solvents and polymers may range from 10 mM to 5M, and is preferably between about 100 mM to about 1M.

For mechanical shearing such as bead disruption to be used as a universal sample preparation approach, it is necessary to characterize and optimize operating parameters with respect to different target material (DNA, RNA or protein) and their source (environmental, blood, or tissue). Although a single system is suitable for disruption different sample types, to optimize results parameters such as power input and the duration of applying sonic agitation may vary with respect to different cell types. Furthermore, it is understood that the concentration of the size stabilizer, the size of the glass beads and the inclusion of enzymes such as collagenase and hyaluronase are all further embodiments of the invention and are no way limiting.

It is understood that magnetic particles, glass beads or a combination of both can be used for disruption without departing from the invention. In one embodiment the magnetic particles are formed of iron oxides. In one embodiment the particles are in the 40-200 nm size range. The particles can be accelerated using an ultrasonic force and can shred the sample. In one embodiment, glass beads are used in the extraction mixture for efficient lysis of spores.

In one embodiment the mechanical force used to release the nucleic acid molecules is sonic vibration accomplished by contacting a container of the fragments suspended in protective buffer with source of sonic vibrations. Such a source may be a commercial ultrasonic transducer or a piezo electric crystal activated by an AC voltage. Such devices are well known to those skilled in the art. Shearing frequencies can be from 10,000 Hz to 10 MHz, preferably between 20 KHz and 4 MHz, and most preferably between 20 KHz and 40 KHz. To assist the shearing of protected nucleic acid molecules samples such as, for example, spores, small beads may be added to the sample. The sonic induced movement of the beads breaks the spore walls to release the nucleic acid molecules contained within. The beads may range in size from about 1 micron to about 1 mm, preferably from about 10 microns to about 500 microns and most preferably from about 50 microns to about 200 microns. The beads may be a metal such as stainless steel, glass or a dense metallic oxide such as zirconium oxide. The time required for shearing the nucleic acid molecules depends partly on the size of the sample and power transmitted from the transducer to the sample. However, when the sheared sample reaches a steady state, which depends on the composition of the protective buffer, there is no further change in the nucleic acid molecules size distribution with further sonication. In practice, sonication times of 15 seconds to 2 minutes at a power level of 1 to 2 watts with a sample size of 100 ul of buffer containing 1 microgram of nucleic acid molecules are sufficient to reach a steady state.

In another embodiment, the sample preparation process further includes the addition of RNase inhibitors to prevent sample degradation. In one embodiment, the sample preparation process includes diethylpyrocarbonate (DEPC), ethylene diamine tetraacetic acid (EDTA), proteinase K, or a combination thereof.

In another embodiment, the presence of a size stabilizer also stabilizes RNA. The SDS and guandinium thiocyanate disrupt the RNAses in the sample thus preserving the RNA.

In one embodiment the magnetic nanoparticle is a magnetite nanoparticle. Magnetite particles are common in nature, and can be collected from beach sands at the edge of the ocean by screening with a magnet. Grinding these particles will produce a relatively coarse magnetic powder. Smaller sized particles can be produced by adding a solution of mixed ferric and ferrous chloride to a stirred aqueous alkaline solution of sodium or ammonium hydroxide. Even smaller sized particles are produced by thermal decomposition of iron acetonylacetonate in dibenzyl ether in the presence of hexadecanediol, oleyl amine and oleic acid. Numerous methods for making magnetite are known. For example, Sun et al. discloses slowly adding a mixture of ferric and ferrous chloride into stirred ammonia. Langmuir, 2009, 25 (10), pp 5969-5973. U.S. Pat. No. 4,698,302 teaches mixing ferrous and ferric chloride with sodium hydroxide. Samanta et al, discloses adding ammonia to a stirred mixture of ferric and ferrous chloride in an inert atmosphere. Journal of Materials Chemistry, 2008, 18, 1204-1208. Duan et al. teaches dissolving iron oxide in oleic acid to form a complex that forms magnetite nanoparticles when heated to 300 degrees C. J. Phys. nucleic acid molecule Chem. C, 2008, 112 (22), pp 8127-8131. Additionally, Yin et al. discloses thermally decomposing iron pentacarbonyl in the presence of oleic acid, Journal of Materials Research, 2004, 19, 1208-1215.

Numerous types of samples can be proceed by applying a mechanical force to break apart the sample to release nucleic acid molecules. The sample preparation process is suitable for use on liquids, solids, soil samples, animal tissue, insect carcasses, DNA, bacterial cells, spores and viruses. As shown in FIG. 3, several disparate samples were processed using identical parameters. Samples of purified DNA, bacterial cells, spores, viruses and fruit flies were all treated using the following technique: each sample was subjected to sonication treatment for two minutes in the presence of magnetic nano-particles and 100 micron glass beads. As shown in FIG. 3, all sample types provided a similar fragment distribution.

As a variety of types of samples can be used, a single system can be used with a wide variety of target organisms without the need to modify the preparation process. Furthermore, even if a sample contains two different targets, nucleic acid molecules can be purified from both components.

The sample preparation system works with small quantities and produces a narrow distribution of nucleic acid molecule fragments for analysis. Optionally, the preparation system passes sample through steps that filter the sample prior to applying a shear force.

FIG. 3, demonstrates that using this process the nucleic acid molecules from a wide variety of different samples can be treated with the same power levels and time of sonication to give the same size distribution of fragments.

In one embodiment, the process further contains the steps necessary to clean the nucleic acid molecules. After release of the nucleic acid molecules and shearing to a useful size range, it is advantageous to clean the nucleic acid molecules from cell debris, proteins, sonication beads and the protection buffer to provide a purified nucleic acid molecule solution in a buffer compatible with subsequent nucleic acid molecule operations and procedures.

In one embodiment, a magnet is utilized to generate an magnetic field. The magnet can pull or push magnetic particles. The magnet can concentrate a sample of magnetic particles or speed up the diffusion process by guiding any magnetic particles.

In one embodiment, magnetic nanoparticles are located in a sample chamber along with a target analyte. The magnetic nanoparticles have an affinity for the target analyte. By attaching the magnetic nanoparticles to the target analyte and applying a magnetic field the target analyte is manipulated to desired locations within the sample chamber.

In one embodiment, a precipitation buffer in solution with the target analyte fragments and the magnetic nanoparticle. The precipitation buffer precipitates the target analyte out of solution and the target analyte is drawn to the magnetic nanoparticles. The precipitation buffer can be any buffer that precipitates the target analyte from the solution. For proteins, the precipitation buffer includes, but is not limited to organic precipitants such as, ammonium sulfate, trichloroacetic acid, acetone, or a mixture of chloroform and methanol. For nucleic acid molecules such as DNA suitable precipitation buffers include, but are not limited to, water miscible organic solvents, acetone, dioxane and tetrahydrofuran. While examples of precipitation buffers are provided, it is understood that any suitable precipitation buffer can be utilized without deflecting from this claimed invention.

In another embodiment, the magnetic nanoparticles contain superparamagnetic particles. The superparamagnetic particles include metal oxides, such as iron oxides. A preferred iron oxide is magnetite (Fe₃O₄).

Once the sample is lysed, the nucleic acid molecules can be magnetically separated from the reminder of the sample. The nucleic acid molecules bind to magnetic particles. In one embodiment, the binding occurs in a high salt/alcohol condition and is eluted using a low salt chelating buffer such as sodium citrate with increased temperature. In one embodiment the sample is heated to at least 60° C. to increase the yield from elution.

Once the magnetic nanoparticles are attached to the target analyte a magnetic field is applied to the reaction chamber. The application of the magnetic field causes the magnetic nanoparticles and any attached target analytes to concentrate in one portion of the reaction chamber. The sample is pulled from the concentrated region of the sample chamber providing a large amount of target analytes comparative the amount of volume extracted. By concentrating the sample more sensitive tests can be preformed.

In another embodiment, the magnetic field holds the magnetic nanoparticle steady as the remaining sample is removed from the chamber. The binding force between the magnetic nanoparticle and the target analyte is sufficient to prevent the target analyte from being removed.

In one embodiment a dispersion of magnetic nanoparticles is added to the sample. The mixture is then incubated at about 60° C. to facilitate the binding. A precipitation buffer is then added to the mixture. The bound complex of nucleic acid molecules and magnetite is then collected in a magnetic field. In one embodiment, the complex is collected on a side wall of the container so any unbound solids can fall to the bottom of the container for easy removal. The buffer and any unbound solids are then removed from the sample.

Optionally, additional rinse steps are used to purify the sample. The cleaning removes compounds which could inhibit binding of nucleic acid molecules. The complex can be washed with additional precipitation buffer, or a washing buffer that does not disturb the complex. After washing, the buffer is drained from the complex resulting in a purified, concentrated sample.

Suitable binding buffers are optionally added to the solution. Binding buffers for the nucleic acid molecule/magnetite complex are, for the most part, buffers in which nucleic acid molecules are insoluble. Precipitation of the nucleic acid molecules promotes binding of the nucleic acid molecules to the magnetite particles. The binding buffer for nucleic acid molecules and magnetite nanoparticles may contain water, sodium acetate, sodium chloride, lithium chloride, ammonium acetate, magnesium chloride, ethanol, propanol, butanol, glycogen or other sugars, polyacrylamide or mixtures thereof. In one embodiment the binding buffer is isopropanol. Binding of the nucleic acid molecules to the magnetite nanoparticles is not instantaneous. In one embodiment the mixture is incubated above room temperature to speed the binding process.

For further processing of the nucleic acid molecules, for some processes, it is necessary to remove the magnetite particles. In one embodiment the nucleic acid molecule is eluted from the complex of nucleic acid molecules and magnetite by heating a mixture of an elution buffer and the complex to 95° C. The magnetite can be collected by a magnetic field, or by centrifugation, providing purified nucleic acid molecules in elution buffer. In one embodiment the elution buffers contain a salt which interacts strongly with iron oxide surfaces. Preferred buffers are phosphate and citrate salt solutions.

### EXAMPLES

### Example 1

### Sonication Bead Disruption

Spores were prepared and isolated from Bacillus subtilis from sporulation media+. To a 100 ul aliquot of the spores taken from the culture, an equal volume of 0.1 mm glass beads were added in a microfuge tube. The tip of the microfuge tube was placed in the socket of a Branson Ultrasonic sonicator. Using a power setting of 2, the beads within the tube were agitated for two minutes. Afterwards, gram staining showed that greater than 90% of the spores were disrupted by this process. This was confirmed with plating assays by counting colonies formed from spores surviving the process. Estimation of the amount of DNA released was accomplished by spotting an aliquot of the lysate onto the surface of a 1% agarose gel containing 1 mg/ml ethidium bromide. A Bio-Rad Fluor-S imager compared the intensity of the sample fluorescence against known standard amounts of DNA also spotted onto the gel surface. Using this technique, approximately 10 ng of DNA can be isolated from 2.5×10⁵ spores.

### Example 2

### Tissue Samples

As shown in FIG. 4, for diagnostic samples, an approach using tissue from the ear of a cow was evaluated. Ear tissue is often taken from cattle for evaluation and has skin, hair, large amounts of cartilage and is rich in blood. Ear plugs of about 3 mm in diameter were tested. A robust sample of about 1 microgram of nucleic acid molecules was isolated from an earplug using ultrasonication and 40 nm ferrite particles. The nucleic acid molecules were in the expected size range. Glass beads were not required for extraction from the tissue and subsequent treatment of an ear plug with bead beating did not result in additional nucleic acid molecule extraction. Sonication power and time settings were identical to those used in the previous examples.

### Example 3

### Samples Contaminated with Soil

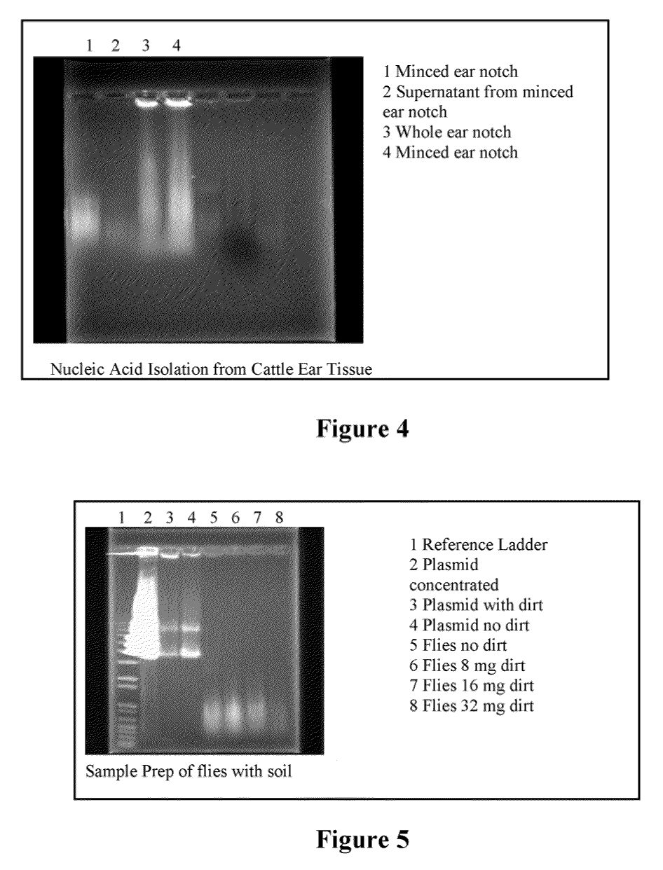

As shown in FIG. 5, to evaluate complex samples, bacterial and spore samples mixed with soil were processed. Soil is a complex medium which is known to inhibit PCR-based systems. Soil was added to samples containing six whole fruit flies. The flies are intended to represent insects that might be evaluated for carrying a disease like malaria. Up to 32 milligrams of the soil were added per milliliter of sample. The fruit flies were disrupted using ultrasonication in the presence of ferrite particles for two minutes. DNA and RNA were captured using ferrite particles with the addition of ethanol. The particles were collected magnetically, washed with buffer and ethanol to remove contaminants then concentrated with magnetics. The nucleic acid molecules were then eluted in hybridization buffer at 90° C. to denature the DNA component. Minimal loss was seen until the level of soil in the sample reached 32 milligrams per 100 micro liters (lane 8) where the solution becomes viscous and particle movement is difficult under the current test conditions. It is understood that by increasing the disrupting power, modifying the solution, or changing the disrupting particles size or characteristics results could be optimized for extremely contaminated samples.

### Example 4

### Preparation of Magnetite Clusters

A first solution of ferric chloride (0.8M), ferrous chloride (0.4M) and hydrochloric acid (0.4M) was mixed and 0.2 micron filtered. A second solution was prepared with 72 ml of ammonium hydroxide (30%) with water to make 1 liter.

1 ml of the ferric/ferrous chloride solution was added with stirring to 20 ml of the ammonium hydroxide solution. Stirring was continued for 15 seconds. The solution (in a 20 ml vial) was placed on a strong magnet and allowed to stand for 1 minute, after which all the product was pulled to the bottom of the vial. The clear supernatant liquid was decanted, replaced with water, mixed, and placed near the magnet. Again the product was pulled to the bottom of the vial. This process was repeated three times to wash the product free from any residual ammonium and iron salts. The vial was then filled with 20 ml of water and ultra-sonicated for 5 minutes at 4 watts power. The suspension was then filtered through a 1 micron glass filter to give a stable suspension of magnetite particles that remain in suspension until pulled down by magnetic forces or centrifugation.

### Example 5

### Attachment of Magnetic Particles

Nucleic acid molecules were purified from fruit flies, then lysed with ferrite particles followed by magnetic separation and elution. The magnetic beads captured more than 90% of available nucleic acid molecules.

### Example 6

### DNA from Complex Samples

Bacillus cells were mixed with cattle ear tissue or whole fruit flies and the mixtures were taken through the sample preparation process. The resulting nucleic acids were hybridized to probes on sensor chips. The chips were then treated with YOYO-1 dye to detect hybridized DNA. The target DNA sequences in the cells hybridized to the sensor chips at levels comparable to Bacillus cells processed separately. Negative controls without Bacillus showed no hybridized DNA. The experiment was repeated with dirt added to the samples as described above. Hybridization efficiency remained at least 60% of the hybridization seen in the sample without eukaryotic cells and dirt.

### Example 7

### Washing Particles with a Flow

Magnetic particles were bound to DNA and then the solution introduced into a clear plastic tube with a 2 mm diameter. A magnet was placed under the center of the tube. A wash buffer was pushed through the tube using a syringe pump. The particles visually remained in place through the washing. After washing the magnet was removed and the particles were rinsed out of the tube. DNA was eluted at high temperature and run on a gel. No apparent loss of DNA was observed.

### Example 8

### Efficiency of Binding and Release of Magnetic Particles

Radiolabeled DNA was used to determine the efficiency of binding to ferrite and the release of the nucleic acid molecules. Radiolabeled DNA with the magnetite suspension and three volumes of ethanol were mixed. The magnetite was pulled to the bottom of the tube using a magnet. The supernatant fluid was removed from the pellet and both fractions were counted in a scintillation counter. The supernatant contained 770 cpm and the resuspended pellet contained 19,330 cpm. Therefore about 96% of the Radiolabeled DNA was bound to the ferrite.

### Example 9

### Release of Nucleic Acid Molecules

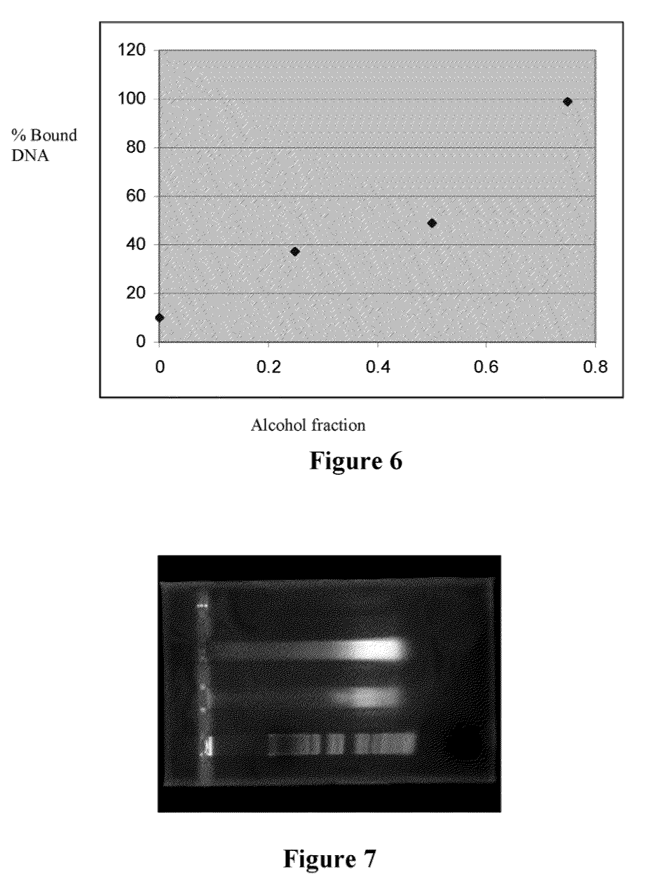

Radiolabeled DNA was used to determine the efficiency of binding to ferrite and the release of the nucleic acid molecules. Radiolabeled DNA with the magnetite suspension and three volumes of ethanol were mixed. The magnetite was pulled to the bottom of the tube using a magnet. The supernatant fluid was removed from the pellet and both fractions were counted in a scintillation counter. Binding was measured as a function of the fraction of ethanol in the mix. The results are shown in FIG. 6.

To determine the release efficiency, the bound DNA pellet was suspended in 100 μl of buffer as indicated in the table below, incubated for 10 minutes at 95° C., then collected on the magnet. The supernatant was separated from the pellet and both were counted.

| Buffer | | Supernatant cpm | Pellet cpm | % Free |
|---|---|---|---|---|
| 500 | mM Phosphate | 43,450 | 1925 | 96% |
| 50 | mM Phosphate | 18,409 | 684 | 96% |
| 60 | mM Citrate | 33,276 | 2164 | 94% |

### Example 10

### DNA from Complex Samples

BG cells were mixed with cattle ear tissue or whole fruit flies and the mixtures were taken through the sample preparation process. The resulting nucleic acids were hybridized to probes on sensor chips. The chips were then treated with YOYO-1 dye to detect hybridized DNA. The target DNA sequences in the cells hybridized to the sensor chips at levels comparable to BG cells processed separately. Negative controls without BG showed no hybridized DNA. The experiment was repeated with dirt added to the samples as described above. Hybridization efficiency remained at least 60% of the hybridization seen in the sample without eukaryotic cells and dirt.

### Example 11

Three fruit flies were placed in each of two 1.5 ml Eppendorf tubes. One was loaded with 100 microliters of a mixture of 100 mM TRIS hydrochloride (pH 7.5), 1.5% dextran sulfate and 0.2% sodium dodecylsulfate (SDS). The other was loaded with 100 microliters of isopropyl alcohol and 10 microliters of 20% sodium dodecylsulfate. Both tubes were loaded with 10 microliters of 0.6% magnetite nanoparticles in water. Both tubes were sonicated at 20 kHz for 45 seconds (2 watts). Then 1 ml of isopropyl alcohol was added to the first tube and ½ ml of isopropyl alcohol was added to the second tube. The magnetic pellet was collected by a permanent magnet, the supernatant liquid decanted and 50 μl of 100 mM sodium phosphate was added to each tube, the pellet resuspended by repetitive pipetting, then incubated at 95 degrees C. for 2 minutes. The pellet was again collected on a magnet and the eluted DNA was run on a 1% agarose gel at 77 volts in TEA buffer. A DNA ladder was also run on the gel.

As shown in FIG. 7, the gel was stained with ethidium bromide and photographed with 302 nm excitation and a 610 nm filter over the camera. The purified DNA is clearly visible on the photograph. The top lane represents the second tube, the middle lane represents the first tube and the bottom lane represents a DNA ladder.

### Example 12

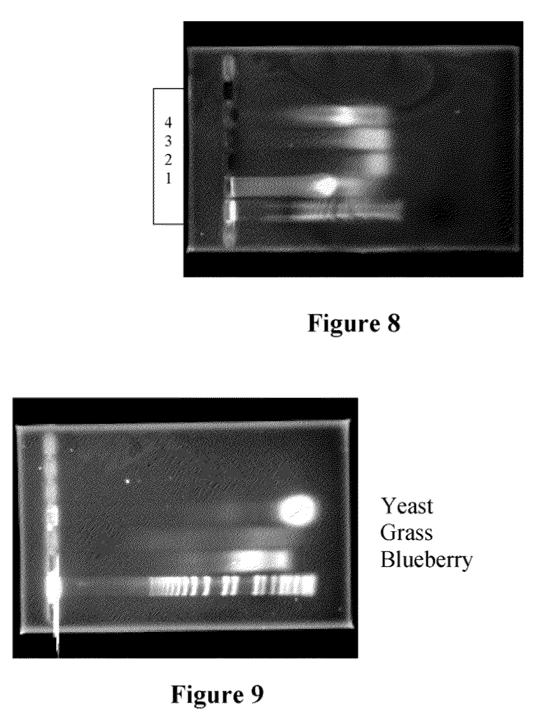

Four tubes, each with three fruit flies, 100 microliters of buffer and 10 μl of 0.6% magnetite nanoparticles were sonicated for 30 seconds at 5 watts at 20 kHz. The DNA was collected, eluted, run on a gel, stained and photographed as in Example 11 and shown in FIG. 8. The four buffers were as follows:

1. 100 mM TRIS, 1.5% Dextran sulfate and 0.2% SDS

2. Isopropylalcohol (IPA)

3. 90% IPA, 1% dodecylbenzenesulfate, 9% water

4. 90% IPA, 1% polyacrylic acid sodium salt, 9% water

### Example 13

Portions of yeast, grass and blueberries were sonicated in 100 mM TRIS,

1.5% Dextran sulfate and 0.2% SDS as in Example 11. The purification, gel and photograph were as in Example 11 and are shown in FIG. 9.

### Example 14

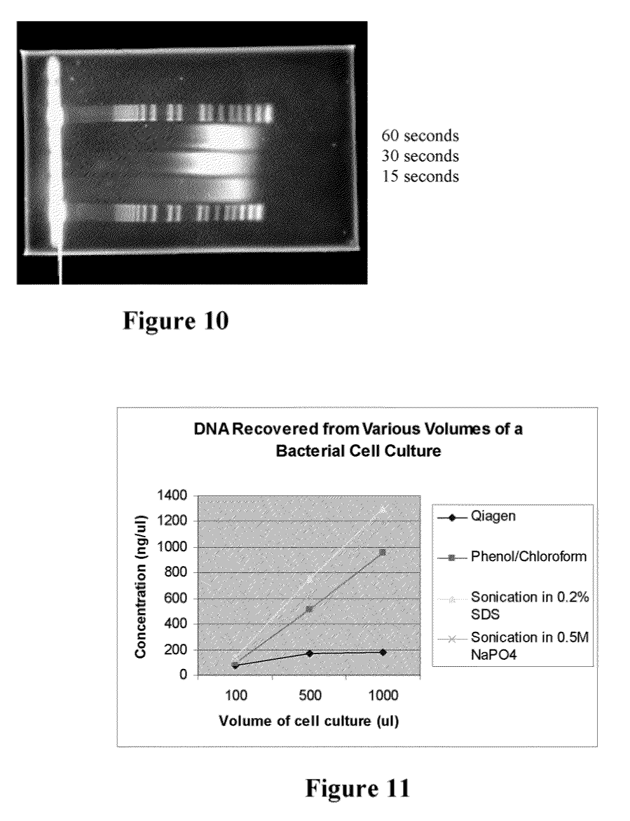

Three 1.5 ml Eppendorf tubes each containing about 10 billion E. coli cells and 33 mg of glass beads (100 micron diameter) and 40 microliters of 0.5 molar sodium phosphate, pH 7.5 were sonicated for 15, 30 and 60 seconds at 40 kHz, 10% amplitude with a 4 mm sonic tip inserted into the tube. The purification, gel and photograph were done as in Example 11 and are shown in FIG. 10.

This example shows that longer sonication times do not change the size distribution, i.e., that steady state conditions apply.

### Example 15

In this example, DNA is recovered from increasing volumes of a bacterial cell culture using two standard methods—the commercial Qiagen kit for DNA recovery and the textbook Phenol/Chloroform method. These were compared to the method given in Example 11, using 0.2% SDS and 0.5 M sodium phosphate as the buffer. The results are shown graphically in FIG. 11.

The graph shows that the method of this invention is superior to both the Qiagen kit and the phenol/chloroform method.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A method for extracting nucleic acid fragments comprising the steps of:
providing a biological sample;
suspending said biological sample in a suspension solution comprising a size stabilizer;
applying a mechanical force to said biological sample to extract nucleic acid molecules;
the application of said mechanical force in the presence of said size stabilizer results in said nucleic acid molecules being reduced to a median fragment size having a median number of base pairs, wherein at least 60% of said nucleic acid molecules have a number of base pairs that are within 50% of said median number of base pairs.

2. The method of claim 1 wherein said mechanical force is a shearing force.

3. The method of claim 1 wherein said mechanical force is sonic vibration.

4. The method of claim 3 wherein said sonic vibration has shearing frequencies between 10,000 Hz and 10 MHz.

5. The method of claim 1, wherein at least 75% of said nucleic acid molecules after the application of said mechanical force have between 200 and 500 base pairs.

6. The method of claim 1, wherein at least 75% of said nucleic acid molecules after the application of said mechanical force have more than 1000 base pairs.

7. The method of claim 1, wherein said suspension solution further comprises disrupting beads.

8. The method of claim 7, wherein said disrupting heads are glass beads.

9. The method of claim 1, wherein said size stabilizer comprises at least one compound selected from the group consisting of sodium phosphate, guanidinium hydrochloride, dextran sulfate sodium dodecyl sulfate, sodium dodceyl benzene sulfate, polyethyleneglycol, anionic surfactants, dipole aprotic solvents, dimethylsulfoxide, dimethyl formamide, dimethylacetamide, hexamethyl phosphoramide, tetramethylurea, kaotropic salt, poly vinyl alcohol, polyethylenimine, poly acrylic acid and other polymeric acids.

10. The method of claim 1, wherein said size stabilizer is selected from the group consisting of sodium dodecyl sulfate and sodium dodceyl benzene sulfate

11. The method of claim 1 further comprising the step of binding the nucleic acid molecules to magnetic nanoparticles.

12. The method of claim 11 further comprising the step of collected said magnetic nanoparticles and the bound nucleic acid molecules and washing magnetic nanoparticles and the bound nucleic acid molecules.

13. The method of claim 12 further comprising the step of eluting said nucleic acid molecule from said magnetic nanoparticles.

14. The method of claim 13 further comprising the step of providing an elution solution comprising alcohol to elute said nucleic acid molecule from said magnetic nanoparticles.

15. The method of claim 11 further comprising the step of applying a magnetic field to concentrate said magnetic nanoparticles and the bound nucleic acid molecules.

16. The method of claim 1, wherein said nucleic acid molecules is reduced to a median number of base pairs between 200 and 10,000 base pairs.

17. The method of claim 1, wherein said size stabilizer limits the minimum size of said nucleic acid molecules achievable by said shearing force to 150 base pairs.

18. The method of claim 1, wherein said biological sample is selected from the group consisting of bacterial cells, spores, viruses and biological tissue.

19. The method of claim 1, wherein said mechanical force is applied via a nebulizer or a homogenizer.

20. The method of claim 1, wherein at least 75% of said nucleic acid molecules have a number of base pairs that are within 50% of the number of base pairs as said median number of base pairs.
